(19) United States
(12) Patent Application Publication
(10) Pub. No.: US 2007/0149514 A1
(43) Pub. Date: Jun. 28, 2007

(54) Indole-phenylsulfonamide derivatives used as ppar-delta activating compounds

(75) Inventors: Michael Woltering, Hilden (DE); Hilmar Bischoff, Wuppertal (DE); Elke Dittrich-Wengenroth, Wuppertal (DE); Heike Heckroth, Wuppertal (DE); Michael Otteneder, Arlesheim (CH)

Correspondence Address: JEFFREY M. GREENMAN, BAYER PHARMACEUTICALS CORPORATION, 400 MORGAN LANE, WEST HAVEN, CT, 06516, US

(73) Assignee: Bayer HealthCare AG, Leverkusen (DE)

(21) Appl. No.: 10/539,707

(22) PCT Filed: Dec. 24, 2003
(86) PCT No.: PCT/EP03/14882

§ 371 Date: Aug. 17, 2006

(30) Foreign Application Priority Data
Jan. 7, 2003 (DE) 10300099.2

(51) Int. Cl. A61K 31/541; A61K 31/5377; A61K 31/496; A61K 31/454; A61K 31/405; C07D 403/02; C07D 413/02; C07D 417/02

(52) U.S. Cl. 514/227.8; 514/232.8; 514/254.09; 514/323; 514/414; 544/60; 544/143; 544/373; 546/201; 548/465

(57) ABSTRACT

The invention relates to novel indole-phenylfulfonamide derivatives, to methods for the production thereof, and to their use in medicaments, particularly as potent PPAR-delta activating compounds, for the prevention and/or treatment of cardiovascular diseases, particularly dyslipidemias and coronary heart diseases.

The present application relates to novel substituted indolephenylsulfonamide derivatives, to processes for their preparation and to their use in medicaments, especially as potent PPAR-delta-activating compounds for the prophylaxis and/or treatment of cardiovascular disorders, especially dyslipidemias and coronary heart diseases.

In spite of many successful therapies, coronary heart diseases (CHDs) remain a serious public health problem. While treatment with statins, by inhibition of HMG-CoA reductase, very successfully lowers the plasma concentration of LDL cholesterol and this leads to a significant lowering in the mortality of patients at risk, there is to date a lack of successful treatment strategies for the therapy of patients having an unfavorable HDL/LDL cholesterol ratio and/or hypertriglyceridemia.

To date, fibrates constitute the only form of therapy for patients of these risk groups. They act as weak agonists of the peroxisome proliferator-activated receptor (PPAR)-alpha (Nature 1990, 347, 645-50). A disadvantage of fibrates which have been approved to date is that their interaction with the receptor is only weak and leads to high daily doses and distinct side effects.

For the peroxisome proliferator-activated receptor (PPAR)-delta (Mol. Endocrinol. 1992, 6, 1634-41), the first pharmacological findings in animal models indicate that potent PPAR-delta agonists may likewise lead to an improvement in the HDL/LDL cholesterol ratio and in hypertriglyceridemia.

WO 00/23407 discloses PPAR modulators for the treatment of obesity, atherosclerosis and/or diabetes. WO 93/15051 and EP 636 608-A1 describe 1-benzenesulfonyl-1,3-dihydroindol-2-one derivatives as vasopressin and/or oxytocin antagonists for the treatment of various disorders. Substituted indolephenylsulfonamide derivatives having antiviral activity are described in WO 01/34146.

It is an object of the present invention to provide novel compounds which can be used as PPAR-delta modulators.

It has now been found that compounds of the general formula (I) 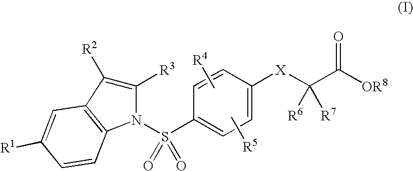
in which
x is O, S or CH₂,

R¹ is (C₆-C₁₀)-aryl or 5- to 10-membered heteroaryl having up to three heteroatoms from the group of N, O and/or S, each of which may be mono- to trisubstituted, identically or differently, by substituents selected from the group of halogen, cyano, nitro, (C₁-C₆)-alkyl which may itself be substituted by hydroxyl or amino, (C₁-C₆)-alkoxy, trifluoromethyl, trifluoromethoxy, (C₂-C₆)-alkenyl, (C₁-C₆)-alkylthio, (C₁-C₆)-alkylsulfonyl, (C₁-C₆)-alkanoyl, (C₁-C₆)-alkoxycarbonyl, hydroxycarbonyl, aminocarbonyl, amino, (C₁-C₆)-acylamino, mono- and di-(C₁-C₆)-alkylamino and 5- to 6-membered heterocyclyl having up to two heteroatoms from the group of N, O and/or S,
R² is phenyl or 5- to 6-membered heteroaryl having up to three heteroatoms from the group of N, O and/or S, each of which may be mono- to trisubstituted, identically or differently, by substituents selected from the group of halogen, cyano, nitro, trifluoromethyl, (C₁-C₄)-alkyl, hydroxyl, trifluoromethoxy and (C₁-C₄)-alkoxy,
   or
   is (C₁-C₆)-alkyl or (C₁-C₆)-alkanoyl, each of which may be substituted by substituents selected from the group of mono- and di-(C₁-C₆)-alkylamino which may itself be substituted by hydroxyl, amino or cyano, and 5- to 6-membered heterocyclyl which has up to two heteroatoms from the group of N, O and/or S and may itself be substituted by (C₁-C₄)-alkyl,
R³ is hydrogen or (C₁-C₄)-alkyl,
R⁴ is hydrogen or (C₁-C₆)-alkyl,
R⁵ is hydrogen, (C₁-C₆)-alkyl, (C₁-C₆)-alkoxy or halogen,
R⁶ and R⁷ are the same or different and are each independently hydrogen or (C₁-C₄)-alkyl,
and
R⁸ is hydrogen or a hydrolyzable group which can be decomposed to the corresponding carboxylic acid,

and the pharmaceutically acceptable salts, solvates and solvates of the salts thereof,

exhibit pharmacological action and can be used as medicaments or for the preparation of medicament formulations.

In the context of the invention, in the definition of R⁸, a hydrolyzable group means a group which, especially in the body, leads to conversion of the —C(O)OR⁸ moiety to the corresponding carboxylic acid (R⁸=hydrogen). Such groups are, for example and with preference: benzyl, (C₁-C₆)-alkyl or (C₃-C₈)-cycloalkyl, each of which is optionally mono- or polysubstituted, identically or differently, by halogen, hydroxyl, amino, (C₁-C₆)-alkoxy, carboxyl, (C₁-C₆)-alkoxycarbonyl, (C₁-C₆)-alkoxycarbonyl-amino or (C₁-C₆)-alkanoyloxy, or in particular (C₁-C₄)-alkyl which is optionally mono- or polysubstituted, identically or differently, by halogen, hydroxyl, amino, (C₁-C₄)-alkoxy, carboxyl, (C₁-C₄)-alkoxycarbonyl, (C₁-C₄)-alkoxycarbonylamino or (C₁-C₄)-alkanoyloxy.

In the context of the invention, (C₁-C₆)-alkyl and (C₁-C₄)-alkyl represent a straight-chain or branched alkyl radical having from 1 to 6 and from 1 to 4 carbon atoms respectively. Preference is given to a straight-chain or branched alkyl radical having from 1 to 4 carbon atoms. Preferred examples include: methyl, ethyl, n-propyl, isopropyl and tert-butyl.

In the context of the invention, (C₂-C₆)-alkenyl represents a straight-chain or branched alkenyl radical having from 2 to 6 carbon atoms. Preference is given to a straight-chain or branched alkenyl radical having from 2 to 4 carbon atoms. Preferred examples include: vinyl, allyl, isopropenyl and n-but-2-en-1-yl.

In the context of the invention, (C₃-C₈)-cycloalkyl represents a monocyclic cycloalkyl group having from 3 to 8 carbon atoms. Preferred examples include: cyclopropyl, cyclobutyl, cyclopentyl and cyclohexyl.

In the context of the invention, (C₆-C₁₀)-aryl represents an aromatic radical having preferably from 6 to 10 carbon atoms. Preferred aryl radicals are phenyl and naphthyl.

In the context of the invention, (C₁-C₆)-alkoxy and (C₁-C₄)-alkoxy represent a straight-chain or branched alkoxy radical having from 1 to 6 and from 1 to 4 carbon atoms respectively. Preference is given to a straight-chain or branched alkoxy radical having from 1 to 4 carbon atoms. Preferred examples include: methoxy, ethoxy, n-propoxy, isopropoxy and tert-butoxy.

In the context of the invention, (C₁-C₆)-alkoxycarbonyl and (C₁-C₄)-alkoxycarbonyl represent a straight-chain or branched alkoxy radical which has from 1 to 6 and from 1 to 4 carbon atoms respectively and is attached via a carbonyl group. Preference is given to a straight-chain or branched alkoxycarbonyl radical having from 1 to 4 carbon atoms. Preferred examples include: methoxycarbonyl, ethoxycarbonyl, n-propoxycarbonyl, isopropoxycarbonyl and tert-butoxycarbonyl.

In the context of the invention, (C₁-C₆)-alkoxycarbonylamino and (C₁-C₄)-alkoxycarbonylamino represent an amino group having a straight-chain or branched alkoxycarbonyl substituent which has from 1 to 6 and from 1 to 4 carbon atoms respectively in the alkoxy radical and is attached via the carbonyl group. Preference is given to an alkoxycarbonylamino radical having from 1 to 4 carbon atoms. Preferred examples include: methoxycarbonylamino, ethoxycarbonylamino, n-propoxycarbonylamino and tert-butoxycarbonylamino.

In the context of the invention, (C₁-C₆)-alkanoyl and (C₁-C₄)-alkanoyl represent a straight-chain or branched alkyl radical which has from 1 to 6 and from 1 to 4 carbon atoms respectively and bears a double-bonded oxygen atom in the 1-position and is bonded via the 1-position. Preference is given to an alkanoyl radical having from 1 to 4 carbon atoms. Preferred examples include: formyl, acetyl, propionyl, n-butyryl, i-butyryl, pivaloyl und n-hexanoyl.

In the context of the invention, (C₁-C₆)-alkanoyloxy and (C₁-C₄)-alkanoyloxy represent a straight-chain or branched alkyl radical which has from 1 to 6 and from 1 to 4 carbon atoms respectively and bears a double-bonded oxygen atom in the 1-position and is attached in the 1-position via a further oxygen atom. Preference is given to an alkanoyloxy radical having from 1 to 4 carbon atoms. Preferred examples include: acetoxy, propionoxy, n-butyroxy, isobutyroxy, pivaloyloxy, n-hexanoyloxy.

In the context of the invention, mono-(C₁-C₆)-alkylamino and mono-(C₁-C₄-alkylamino represent an amino group having a straight-chain or branched alkyl substituent which has from 1 to 6 and from 1 to 4 carbon atoms respectively. Preference is given to a straight-chain or branched monoalkylamino radical having from 1 to 4 carbon atoms. Preferred examples include: methylamino, ethylamino, n-propylamino, isopropylamino and tert-butylamino.

In the context of the invention, di-(C₁-C₆)-alkylamino and di-(C₁-C₄)-alkylamino represent an amino group having two identical or different straight-chain or branched alkyl substituents having in each case from 1 to 6 and from 1 to 4 carbon atoms respectively. Preference is given to straight-chain or branched dialkylamino radicals having in each case from 1 to 4 carbon atoms. Preferred examples include: N,N-dimethylamino, N,N-diethylamino, N-ethyl-N-methylamino, N-methyl-N-n-propylamino, N-isopropyl-N-n-propylamino, N-tert-butyl-N-methylamino, N-ethyl-N-n-pentylamino and N-n-hexyl-N-methylamino.

In the context of the invention, (C₁-C₆)-acylamino represents an amino group having a straight-chain or branched alkanoyl substituent which has from 1 to 6 carbon atoms and is bonded via the carbonyl group. Preference is given to an acylamino radical having from 1 to 2 carbon atoms. Preferred examples include: formamido, acetamido, propionamido, n-butyramido and pivaloylamido.

In the context of the invention, (C₁-C₆)-alkylthio represents a straight-chain or branched alkylthio radical having from 1 to 6 carbon atoms. Preference is given to a straight-chain or branched alkylthio radical having from 1 to 4 carbon atoms. Preferred examples include: methylthio, ethylthio, n-propylthio, isopropylthio, t-butylthio, n-pentylthio and n-hexylthio.

In the context of the invention, (C₁-C₆)-alkylsulfonyl represents a straight-chain or branched alkylsulfonyl radical having from 1 to 6 carbon atoms. Preference is given to a straight-chain or branched alkylsulfonyl radical having from 1 to 4 carbon atoms. Preferred examples include: methylsulfonyl, ethylsulfonyl, n-propylsulfonyl, isopropylsulfonyl, t-butylsulfonyl, n-pentylsulfonyl and n-hexylsulfonyl.

In the context of the invention, 5- to 10-membered and 5- to 6-membered heteroaryl having, respectively, up to 3 and up to 2 identical or different heteroatoms from the group of S, N and/or O respectively preferably represent a mono- or optionally bicyclic aromatic heterocycle (heteroaromatic) which is attached via a ring carbon atom of the heteroaromatic or, if appropriate, via a ring nitrogen atom of the heteroaromatic. Examples include: furanyl, pyrrolyl, thienyl, pyrazolyl, imidazolyl, thiazolyl, oxazolyl, isoxazolyl, isothiazolyl, pyridyl, pyrimidinyl, pyridazinyl, pyrazinyl, benzofuranyl, benzothienyl, benzimidazolyl, benzoxazolyl, indolyl, indazolyl, quinolinyl, isoquinolinyl, naphthyridinyl, quinazolinyl, quinoxalinyl. Preference is given to 5- to 6-membered heteroaryl radicals having up to two heteroatoms from the group of N, O and/or S, for example furyl, thienyl, thiazolyl, oxazolyl, isothiazolyl, isoxazolyl, imidazolyl, pyridyl, pyrimidinyl, pyridazinyl, pyrazinyl.

In the context of the invention, 5- to 6-membered heterocyclyl having up to 2 hetero-atoms from the group of N, O and/or S represents a saturated heterocycle which is bonded via a ring carbon atom or, if appropriate, via a ring nitrogen atom of the heterocycle. Preferred examples include: tetrahydrofuryl, pyrrolidinyl, piperidinyl, piperazinyl, morpholinyl and thiomorpholinyl.

In the context of the invention, halogen includes fluorine, chlorine, bromine and iodine. Preference is given to chlorine or fluorine.

Depending on the substitution pattern, the inventive compounds can exist in stereoisomeric forms which either behave like image and mirror image (enantiomers) or do not behave like image and mirror image (diastereomers). The invention relates both to the enantiomers or diastereomers and to their respective mixtures. The racemic forms, like the diastereomers, can be separated in a known manner into the stereoisomerically uniform constituents.

Furthermore, certain compounds can be present in tautomeric forms. This is known to those skilled in the art, and such compounds are likewise encompassed by the scope of the invention.

The compounds according to the invention can also be present as salts. In the context of the invention, preference is given to physiologically acceptable salts.

Physiologically acceptable salts can be salts of the inventive compounds with inorganic or organic acids. Preference is given to salts with inorganic acids, for example hydrochloric acid, hydrobromic acid, phosphoric acid or sulfuric acid, or to salts with organic carboxylic or sulfonic acids, for example acetic acid, propionic acid, maleic acid, fumaric acid, malic acid, citric acid, tartaric acid, lactic acid, benzoic acid, or methanesulfonic acid, ethanesulfonic acid, benzenesulfonic acid, toluenesulfonic acid or naphthalenedisulfonic acid.

Physiologically acceptable salts can also be salts of the inventive compounds with bases, for example metal or ammonium salts. Preferred examples are alkali metal salts (e.g. sodium salts or potassium salts), alkaline earth metal salts (e.g. magnesium salts or calcium salts), and also ammonium salts which are derived from ammonia or organic amines, for example ethylamine, di- or triethylamine, ethyldiisopropylamine, monoethanolamine, di- or triethanolamine, dicyclohexylamine, dimethylaminoethanol, dibenzylamine, N-methylmorpholine, dihydroabietylamine, 1-ephenamine, N-methylpiperidine, arginine, lysine, ethylenediamine or 2-phenylethylamine.

The inventive compounds and salts thereof can also be present in the form of their solvates, in particular in the form of their hydrates.

Preference is given to compounds of the general formula (I) in which
X is O or S,
R¹ is phenyl or 5- to 6-membered heteroaryl having up to two heteroatoms from the group of N, O and/or S, each of which may be mono- to disubstituted, identically or differently, by substituents selected from the group of fluorine, chlorine, bromine, cyano, (C₁-C₄)-alkyl, (C₁-C₄)-alkoxy, trifluoromethyl, trifluoromethoxy, methylthio, acetyl, (C₁-C₄)-alkoxycarbonyl, amino, mono- and di-(C₁-C₄)-alkylamino,
R² is phenyl, thiazolyl, oxazolyl, isothiazolyl, isoxazolyl, furyl or thienyl, each of which may be mono- to disubstituted, identically or differently, by substituents selected from the group of fluorine, chlorine, bromine, cyano, nitro, trifluoromethyl, methyl, hydroxyl, methoxy and trifluoromethoxy,
   or
   is (C₁-C₄)-alkyl or (C₁-C₄)-alkanoyl, each of which may be substituted by substituents selected from the group of di-(C₁-C₄)-alkylamino, pyrrolidino, piperidino, morpholino, thiomorpholino and piperazino, where the heterocycles mentioned may themselves be substituted by (C₁-C₄)-alkyl,
R³ is hydrogen or methyl,
R⁴ is hydrogen or methyl,
R⁵ is hydrogen, (C₁-C₄)-alkyl, (C₁-C₄)-alkoxy, fluorine or chlorine,
R⁶ and R⁷ are the same or different and are each independently hydrogen or methyl,
and
R⁸ is hydrogen.

Particular preference is given to compounds of the general formula (I) in which
x is O,
R¹ is phenyl which may be mono- to disubstituted, identically or differently, by substituents selected from the group of fluorine, chlorine, methyl, tert-butyl, methoxy, trifluoromethyl, trifluoromethoxy, methylthio and dimethylamino,
R² is thiazolyl, (C₁-C₄)-alkyl, acetyl or a group of the formula —CH₂NR⁹R¹⁰ where
   R⁹ and R¹⁰ are the same or different and are each (C₁-C₄)-alkyl, or, together with the nitrogen atom to which they are bonded, form a pyrrolidine, piperidine, morpholine, thiomorpholine, piperazine or N′-methylpiperazine ring,
R³ is hydrogen,
R⁴ is hydrogen or methyl,
R⁵ is methyl,
R⁶ and R⁷ are each hydrogen,
and
R⁸ is hydrogen.

Of very particular significance are compounds of the formula (I-A) 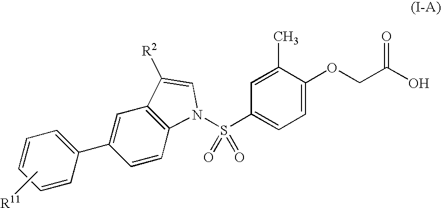
in which
R² is thiazolyl, (C₁-C₄)-alkyl, acetyl or a group of the formula —CH₂NR⁹R¹⁰ where
   R⁹ and R¹⁰ are the same or different and are each (C₁-C₄)-alkyl, or, together with the nitrogen atom to which they are bonded, form a pyrrolidine, piperidine, morpholine, thiomorpholine, piperazine or N′-methylpiperazine ring,
and
R¹¹ is fluorine, chlorine, methyl, tert-butyl, trifluoromethyl, methoxy or trifluoromethoxy.

The radical definitions listed above, in general or specified within areas of preference, applied both to the end products of the formula (I) or (I-A) and correspondingly to the starting materials and intermediates required for the preparation in each case.

Moreover, a process has been found for preparing the inventive compounds, characterized in that

compounds of the general formula (II)

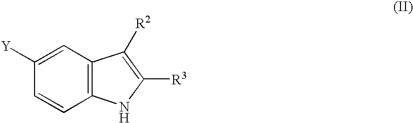

in which R² and R³ are each as defined above and

Y is chlorine or bromine,

are converted initially using a compound of the general formula (III)

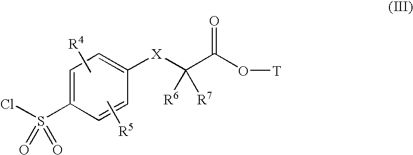

in which X, R⁴, R⁵, R⁶ and R⁷ are each as defined above and

T is benzyl or (C₁-C₆)-alkyl,

in an inert solvent in the presence of a base to compounds of the general formula (IV)

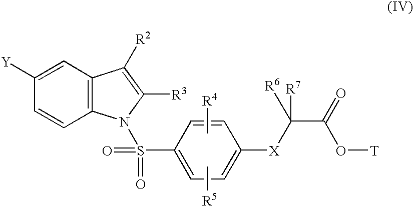

in which T, X, Y, R², R³, R⁴, R⁵, R⁶ and R⁷ are each as defined above,

then the latter are reacted in a coupling reaction with a compound of the general formula (V)

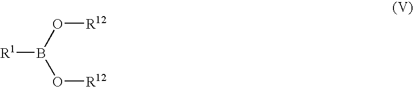

in which R¹ is as defined above and
R¹² is hydrogen or methyl, or both radicals together form a —CH₂CH₂— or —C(CH₃)₂—C(CH₃)₂— bridge,

in an inert solvent in the presence of a suitable palladium catalyst and of a base to give compounds of the general formula (I-B)

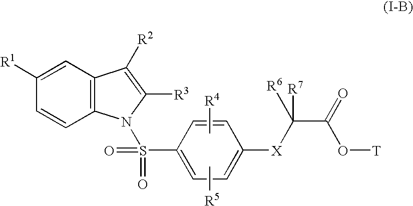

in which T, X, R¹, R², R³, R⁴, R⁵, R⁶ and R⁷ are each as defined above

[cf. for example, W. Hahnfeld, M. Jung, Pharmazie 1994, 49, 18-20; idem, Liebigs Ann. Chem. 1994, 59-64],

then the compounds (I-B) are reacted with acids or bases or, in the case that T is benzyl, also hydrogenolytically to give the corresponding carboxylic acids of the general formula (I-C)

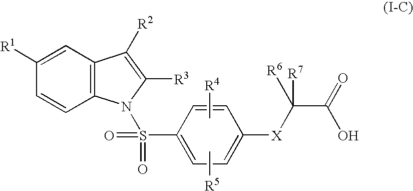

in which X, R¹, R², R³, R⁴, R⁵, R⁶ and R⁷ are each as defined above,

and the carboxylic acids (I-C) are optionally modified by known methods for esterification further to give compounds of the general formula (I).

The coupling reaction step [cf. (IV)+(V)→(I-B)] and the ester cleavage [cf. (I-B)→(I-C)] may optionally also be effected in reverse order in the above-described reaction sequence; it is equally possible to carry out a basic ester cleavage in situ in the course of the coupling reaction.

Inert solvents for the process step (II)+(III)→(IV) are, for example, halohydrocarbons such as dichloromethane, trichloromethane, tetrachloromethane, trichloroethane, tetrachloroethane, 1,2-dichloroethane or trichloroethylene, ethers such as diethyl ether, dioxane, tetrahydrofuran, glycol dimethyl ether or diethylene glycol dimethyl ether, hydrocarbons such as benzene, xylene, toluene, hexane, cyclohexane or mineral oil fractions, or other solvents such as nitromethane, ethyl acetate, acetone, 2-butanone, dimethylformamide, dimethyl sulfoxide, acetonitrile, N-methylpyrrolidinone or pyridine. It is equally possible to use mixtures of the solvents mentioned. Preference is given to dichloromethane, tetrahydrofuran or 2-butanone.

Suitable bases for the process step (II)+(III)→(IV) are the customary inorganic or organic bases. These preferably include alkali metal hydroxides, for example lithium hydroxide, sodium hydroxide or potassium hydroxide, alkali metal or alkaline earth metal carbonates such as sodium carbonate, potassium carbonate or calcium carbonate, alkali metal hydrides such as sodium hydride, or organic amines such as pyridine, triethylamine, ethyldiisopropylamine, N-methylmorpholine or N-methylpiperidine. Particular preference is given to potassium carbonate or amine bases such as triethylamine, pyridine or ethyldiisopropylamine, optionally in the presence of catalytic amounts (approx. 10 mol %) of 4-N,N-dimethylaminopyridine or 4-pyrrolidinopyridine.

In this reaction, the base is used in an amount of from 1 to 5 mol, preferably of from 1 to 2.5 mol, based on 1 mol of the compound of the general formula (III).

The reaction is effected generally within a temperature range of from 0° C. to +150° C., preferably of from +25° C. to +100° C. The reaction may be carried out at standard, elevated or at reduced pressure (for example of from 0.5 to 5 bar). In general, standard pressure is used.

Inert solvents for the process step (IV)+(V)→(I-B) are, for example, ethers such as diethyl ether, dioxane, tetrahydrofuran, glycol dimethyl ether or diethylene glycol dimethyl ether, alcohols such as methanol, ethanol, n-propanol, isopropanol, n-butanol or tert-butanol, hydrocarbons such as benzene, xylene, toluene, hexane, cyclohexane or mineral oil fractions, or other solvents such as dimethylformamide, acetonitrile or else water. It is equally possible to use mixtures of the solvents mentioned. Preference is given to toluene, dimethylformamide or acetonitrile.

Suitable bases for the process step (IV)+(V)→(I-B) are the customary inorganic or organic bases. These preferably include alkali metal hydroxides, for example lithium hydroxide, sodium hydroxide or potassium hydroxide, alkali metal or alkaline earth metal carbonates such as sodium carbonate, potassium carbonate or calcium carbonate, alkali metal phosphates such as sodium phosphate or potassium phosphate, or organic amines such as pyridine, triethylamine, ethyldiisopropylamine, N-methylmorpholine or N-methylpiperidine. Particular preference is given to sodium carbonate or potassium carbonate or potassium phosphate.

In this reaction, the base is used in an amount of from 1 to 5 mol, preferably of from 2 to 3 mol, based on 1 mol of the compound of the general formula (IV).

Suitable palladium catalysts for the process step (IV)+(V)→(I-B) are preferably palladium(0) or palladium(II) compounds which are used preformed, for example [1,1′-bis(diphenylphosphino)ferrocenyl]palladium(II) chloride or bis(triphenylphosphine)palladium(II) chloride, or which can be generated in situ from a suitable palladium source, for example bis(dibenzylideneacetone)palladium(0) or tetrakis(triphenylphosphine)palladium(0), and a suitable phosphine ligand.

The reaction is effected generally within a temperature range of from 0° C. to +150° C., preferably of from +20° C. to +100° C. The reaction may be carried out at standard, elevated or at reduced pressure (for example of from 0.5 to 5 bar). In general, standard pressure is used.

Inert solvents for the process step (I-B)→(I-C) are, for example, halohydrocarbons such as dichloromethane, 1,2-dichloroethane or trichloroethylene, ethers such as diethyl ether, dioxane, tetrahydrofuran, glycol dimethyl ether or diethylene glycol dimethyl ether, alcohols such as methanol, ethanol, n-propanol, isopropanol, n-butanol or tert-butanol, hydrocarbons such as benzene, xylene, toluene, hexane, cyclohexane or mineral oil fractions, or other solvents such as nitromethane, acetone, dimethylformamide, dimethyl sulfoxide, acetonitrile or N-methylpyrrolidinone. It is equally possible to use mixtures of the solvents mentioned. Preference is given to alcohols such as methanol or ethanol.

Suitable bases for the process step (I-B)→(I-C) are the customary inorganic bases. These preferably include alkali metal hydroxides, for example lithium hydroxide, sodium hydroxide or potassium hydroxide, or alkali metal or alkaline earth metal carbonates such as sodium carbonate, potassium carbonate or calcium carbonate. Particular preference is given to lithium hydroxide or sodium hydroxide.

In this reaction, the base is used in amount of from 1 to 5 mol, preferably of from 1 to 3 mol, based on 1 mol of the compound of the general formula (I-B).

Suitable acids for the process step (I-B)→(I-C) are the customary inorganic acids, for example hydrochloric acid or sulfuric acid, or sulfonic acids such as toluenesulfonic acid, methanesulfonic acid or trifluoromethanesulfonic acid, or carboxylic acids such as trifluoroacetic acid.

The reaction is effected generally within a temperature range of from −20° C. to +100° C., preferably of from 0° C. to +30° C. The reaction may be carried out at standard, elevated or at reduced pressure (for example of from 0.5 to 5 bar). In general, standard pressure is used.

The compounds of the general formula (II) are known or may be prepared in analogy to literature processes, for example by converting compounds of the general formula (VI)

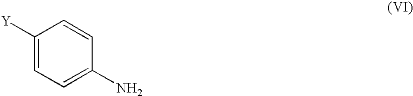

in which Y is as defined above

initially using sodium nitrite and tin(II) chloride in the presence of an acid to hydrazine derivatives of the general formula (VII)

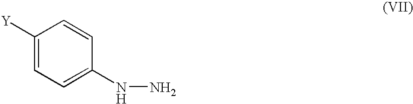

in which Y is as defined above,

and subsequently reacting the latter in the presence of an acid or Lewis acid, optionally in an inert solvent, with a compound of the general formula (VIII)

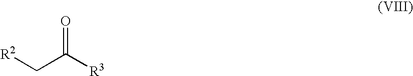

in which R² and R³ are each as defined above.

Inert solvents for the process step (VI)→(VIII) are, for example, ethers such as dioxane, glycol dimethyl ether or diethylene glycol dimethyl ether, alcohols such as methanol, ethanol, n-propanol, isopropanol, n-butanol or tert-butanol, or other solvents such as dimethylformamide, dimethyl sulfoxide, N-methylpyrrolidinone or water. It is equally possible to use mixtures of the solvents mentioned. The preferred solvent is water.

Suitable acids for the process step (VI)→(VII) are the customary inorganic or organic acids. These preferably include hydrochloric acid, sulfuric acid or phosphoric acid, or carboxylic acids such as formic acid, acetic acid or trifluoroacetic acid, or sulfonic acids such as toluenesulfonic acid, methanesulfonic acid or trifluoromethanesulfonic acid. Particular preference is given to semiconcentrated to concentrated aqueous hydrochloric acid which serves simultaneously as the solvent.

The reaction is effected generally within a temperature range of from −30° C. to +80° C., preferably of from −10° C. to +25° C. The reaction may be carried out at standard, elevated or at reduced pressure (for example of from 0.5 to 5 bar). In general, standard pressure is used.

Inert solvents for the process step (VII)+(VIII)→(II) are, for example, halohydrocarbons such as dichloromethane, trichloromethane, tetrachloromethane, trichloroethane, tetrachloroethane, 1,2-dichloroethane or trichloroethylene, ethers such as dioxane, tetrahydrofuran, glycol dimethyl ether or diethylene glycol dimethyl ether, alcohols such as methanol, ethanol, n-propanol, isopropanol, n-butanol or tert-butanol, or hydrocarbons such as benzene, xylene, toluene, hexane, cyclohexane or mineral oil fractions, or other solvents such as acetonitrile or water. It is equally possible to use mixtures of the solvents mentioned. It is also possible to carry out the reaction without solvent. Preference is given to carrying out the reaction without solvent.

Suitable acids for the process step (VII)+(VIII)→(II) are the customary inorganic or organic acids. These preferably include hydrochloric acid, sulfuric acid or phosphoric acid, or carboxylic acids such as formic acid, acetic acid or trifluoroacetic acid, or sulfonic acids such as toluenesulfonic acid, methanesulfonic acid or trifluoromethanesulfonic acids. Alternatively, the customary Lewis acids are also suitable, for example boron trifluoride, aluminum trichloride or zinc chloride. In this reaction, the acid is used in an amount of from 1 to 10 mol, based on 1 mol of the compound of the general formula (VII). The use of zinc chloride, preferably in an amount of from 1 to 2 mol based on 1 mol of the compound (VII), is preferred.

The reaction is effected generally within a temperature range of from +20° C. to +250° C., preferably within a temperature range of from +130° C. to +200° C. The reaction may be carried out at standard, elevated or at reduced pressure (for example of from 0.5 to 5 bar). In general, standard pressure is used.

The compounds of the general formula (III) are known or can be prepared in analogy to literature processes, for example by converting a compound of the general formula (IX)

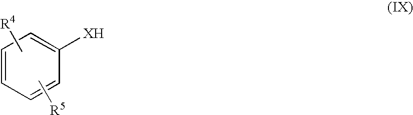

in which R⁴, R⁵ and X are each as defined above

initially using a compound of the general formula (X)

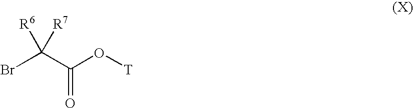

in which R⁶, R⁷ and T are each as defined above

in an inert solvent in the presence of a base to a compound of the general formula (XI)

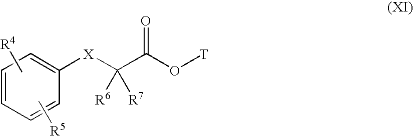

in which R⁴, R⁵, R⁶, R⁷, X and T are each as defined above,

and then reacting the latter with chlorosulfonic acid [cf., for example, P. D. Edwards, R. C. Mauger, K. M. Cottrell, F. X. Morris, K. K. Pine, M. A. Sylvester, C. W. Scott, S. T. Furlong, Bioorg. Med. Chem. Lett. 2000, 10, 2291-2294].

Inert solvents for the process step (IX)+(X)→(XI) are, for example, ethers such as diethyl ether, dioxane, tetrahydrofuran, glycol dimethyl ether or diethylene glycol dimethyl ether, hydrocarbons such as benzene, xylene, toluene, hexane, cyclohexane or mineral oil fractions, or other solvents such as acetone, dimethylformamide, dimethylsulfoxide, acetonitrile or N-methylpyrrolidinone. It is equally possible to use mixtures of the solvents mentioned. Preference is given to dimethylformamide or acetone.

Suitable bases for the process step (IX)+(X)→(XI) are the customary inorganic or organic bases. These preferably include alkali metal hydroxides, for example lithium hydroxide, sodium hydroxide or potassium hydroxide, alkali metal or alkaline earth metal carbonates such as sodium carbonate, potassium carbonate or calcium carbonate, alkali metal hydrides such as sodium hydride, or organic amines such as pyridine, triethylamine, ethyldiisopropylamine, N-methylmorpholine or N-methylpiperidine. Particular preference is given to potassium carbonate.

In this reaction, the base is used in an amount of from 1 to 5 mol, preferably of from 1 to 2 mol, based on 1 mol of the compound of the general formula (IX).

The reaction is effected within a temperature range of from −20° C. to +150° C., preferably of from 0° C. to +80° C. The reaction may be carried out at standard, elevated or at reduced pressure (for example of from 0.5 to 5 bar). In general, standard pressure is used.

The compounds of the general formulae (V), (VI), (VIII), (IX) and (X) are commercially available, known from the literature or can be prepared in analogy to literature processes.

The inventive compounds of the general formulae (I) and (I-A) in which

R² is a group of the formula —CH₂NR⁹R¹⁰ where
R⁹ and R¹⁰ are the same or different and are each (C₁-C₄)-alkyl or, together with the nitrogen atom to which they are bonded, form a pyrrolidine, piperidine, morpholine, thiomorpholine, piperazine or N′-methylpiperazine ring,

and

R³ is hydrogen

may also be prepared by converting compounds of the general formula (XII)

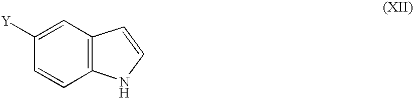

in which Y is as defined above

initially in analogy to literature processes [for example W. Zhang, M. LoCurcio, C.-C. Lin, L. S. Jimenez, J. Heterocycl. Chem. 1996, 33, 1647-1652] by reacting with dichloromethyl methyl ether in the presence of tin tetrachloride to give compounds of the general formula (XIII)

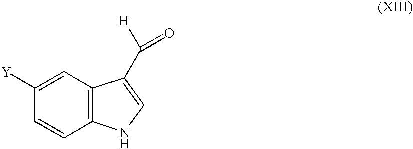

in which Y is as defined above,

then reacting the latter with a compound of the formula (V), analogously to the above-described coupling reaction (IV)+(V)→(I-B), to give compounds of the general formula (XIV)

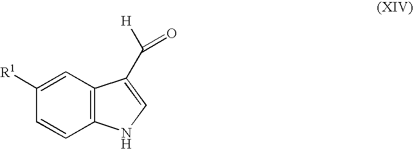

in which R¹ is as defined above,

then converting using a compound of the formula (III), analogously to the above-described reaction (II)+(III)→(IV), to compounds of the general formula (XV)

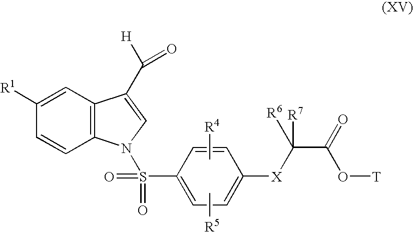

in which T, X, R¹, R⁴, R⁵, R⁶ and R⁷ are each as defined above,

then reacting in the presence of a suitable reducing agent, for example sodium cyanoborohydride or sodium triacetoxyborohydride, with a compound of the general formula (XVI)

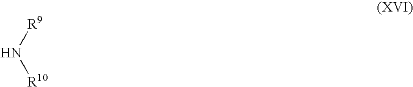

in which R⁹ and R¹⁰ are each as defined above

to give compounds of the general formula (XVII)

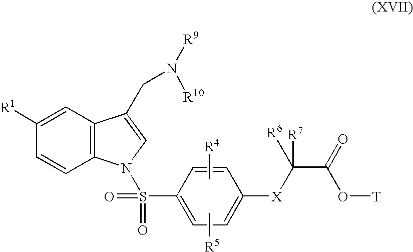

in which T, X, R¹, R⁴, R⁵, R⁶, R⁷, R⁹ and R¹⁰ are each as defined above,

and finally converting using acids or bases or, in the case that T is benzyl, also hydrogenolytically to give the corresponding carboxylic acids of the general formula (I-D)

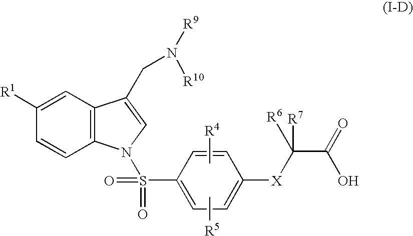

in which X, R¹, R⁴, R⁵, R⁶, R⁷, R⁹ and R¹⁰ are each as defined above.

Inert solvents for the process step (XIII)+(V)→(XIV) are, for example, ethers such as diethyl ether, dioxane, tetrahydrofuran, glycol dimethyl ether or diethylene glycol dimethyl ether, alcohols such as methanol, ethanol, n-propanol, isopropanol, n-butanol or tert-butanol, hydrocarbons such as benzene, xylene, toluene, hexane, cyclohexane or mineral oil fractions, or other solvents such as dimethylformamide, acetonitrile or else water. It is equally possible to use mixtures of the solvents mentioned. Preference is given to toluene, dimethylformamide or acetonitrile.

Suitable bases for the process step (XIII)+(V)→(XIV) are the customary inorganic or organic bases. These preferably include alkali metal hydroxides, for example lithium hydroxide, sodium hydroxide or potassium hydroxide, alkali metal or alkaline earth metal carbonates such as sodium carbonate, potassium carbonate or calcium carbonate, alkali metal phosphates such as sodium phosphate or potassium phosphate, or organic amines such as pyridine, triethylamine, ethyldiisopropylamine, N-methylmorpholine or N-methylpiperidine. Particular preference is given to sodium carbonate or potassium carbonate or potassium phosphate.

In this reaction, the base is used in an amount of from 1 to 5 mol, preferably of from 2 to 3 mol, based on 1 mol of the compound of the general formula (XIII).

Suitable palladium catalysts for the process step (XIII)+(V)→(XIV) are preferably palladium(0) or palladium(II) compounds which are used preformed, for example [1,1′-bis(diphenylphosphino)ferrocenyl]palladium(II) chloride or bis(triphenylphosphine)palladium(II) chloride, or which can be generated in situ from a suitable palladium source, for example bis(dibenzylideneacetone)palladium(0) or tetrakis(triphenylphosphine)palladium(0), and a suitable phosphine ligand.

The reaction is effected generally within a temperature range of from 0° C. to +150° C., preferably of from +20° C. to +100° C. The reaction may be carried out at standard, elevated or at reduced pressure (for example of from 0.5 to 5 bar). In general, standard pressure is used.

Inert solvents for the process step (XIV)+(III)→(XV) are, for example, halohydrocarbons such as dichloromethane, trichloromethane, tetrachloromethane, trichloroethane, tetrachloroethane, 1,2-dichloroethane or trichloroethylene, ethers such as diethyl ether, dioxane, tetrahydrofuran, glycol dimethyl ether or diethylene glycol dimethyl ether, hydrocarbons such as benzene, xylene, toluene, hexane, cyclo-hexane or mineral oil fractions, or other solvents such as nitromethane, ethyl acetate, acetone, 2-butanone, dimethylformamide, dimethyl sulfoxide, acetonitrile, N-methyl-pyrrolidinone or pyridine. It is equally possible to use mixtures of the solvents mentioned. Preference is given to dichloromethane, tetrahydrofuran or 2-butanone.

Suitable bases for the process step (XIV)+(III)→(XV) are the customary inorganic or organic bases. These preferably include alkali metal hydroxides, for example lithium hydroxide, sodium hydroxide or potassium hydroxide, alkali metal or alkaline earth metal carbonates such as sodium carbonate, potassium carbonate or calcium carbonate, alkali metal hydrides such as sodium hydride, or organic amines such as pyridine, triethylamine, ethyldiisopropylamine, N-methylmorpholine or N-methylpiperidine. Particular preference is given to potassium carbonate or amine bases such as triethylamine, pyridine or ethyldiisopropylamine, optionally in the presence of catalytic amounts (approx. 10 mol %) of 4-N,N-dimethylaminopyridine or 4-pyrrolidinopyridine.

In this reaction, the base is used in an amount of from 1 to 5 mol, preferably of from 1 to 2.5 mol, based on 1 mol of the compound of the general formula (III).

The reaction is effected generally within a temperature range of from 0° C. to +150° C., preferably of from +25° C. to +100° C. The reaction may be carried out at standard, elevated or at reduced pressure (for example of from 0.5 to 5 bar). In general, standard pressure is used.

The reaction (XV)+(XVI)→(XVII) is effected in the solvents which are inert under the reaction conditions and are customary for a reductive amination, optionally in the presence of an acid, for example acetic acid, and/or of a dehydrating agent, for example sodium sulfate, magnesium sulfate or molecular sieve. The customary solvents include, for example, ethers such as diethyl ether, dioxane, tetrahydrofuran or glycol dimethyl ether, alcohols such as methanol, ethanol, n-propanol, isopropanol, n-butanol or tert-butanol, halohydrocarbons such as dichloromethane, 1,2-dichloroethane, trichloromethane or tetrachloromethane, or hydrocarbons such as benzene, toluene, xylene, hexane, cyclohexane or mineral oil fractions. It is equally possible to use mixtures of the solvents mentioned. Preference is given to methanol, dichloromethane, 1,2-dichloroethane or trichloromethane, if appropriate with addition of acetic acid.

Suitable reducing agents for the reaction (XV)+(XVI)→(XVII) are complex aluminum hydrides or borohydrides, for example diisobutylaluminum hydride, sodium borohydride, sodium triacetoxyborohydride, sodium cyanoborohydride or tetrabutylammonium borohydride. Preference is given to sodium triacetoxyborohydride.

In this reaction, the reducing agent is used in an amount of from 1 to 5 mol, preferably of from 1 to 2 mol, based on 1 mol of the compound of the general formula (XV). The amine of the general formula (XVI) is preferably used in an amount of from 1 to 2 mol based on 1 mol of the compound (XV).

The reaction is effected generally within a temperature range of from 0° C. to +100° C., preferably of from +20° C. to +80° C. The reaction may be carried out at standard, elevated or at reduced pressure (for example of from 0.5 to 5 bar). In general, standard pressure is used.

Solvents and bases or acids suitable for the process step (XVII)→(I-D) correspond to those mentioned above for the process step (I-B)→(I-C).

The compounds of the general formulae (XII) and (XVI) are commercially available, known from the literature or can be prepared in analogy to literature processes.

The process according to the invention can be illustrated by the following reaction schemes 1 and 2:

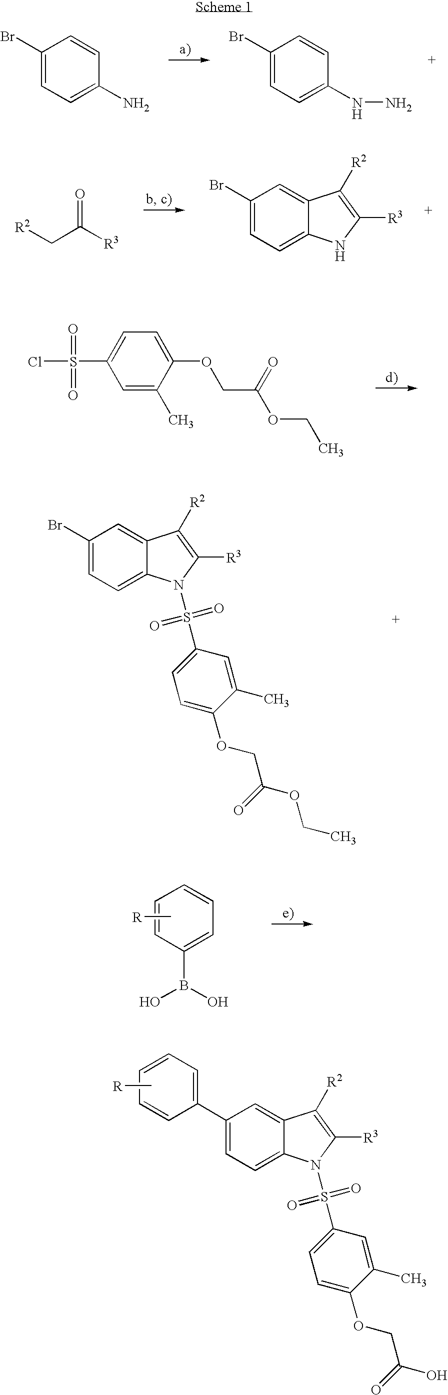

a) NaNO₂, SnCl₂, HCl; b) CH₃CH₂OH, RT; c) ZnCl₂, 170° C., 30 min; d) K₂CO₃, 2-butanone, reflux; e) Pd(PPh₃)₂Cl₂, DMF, aq. Na₂CO₃, 100° C., 15 h.

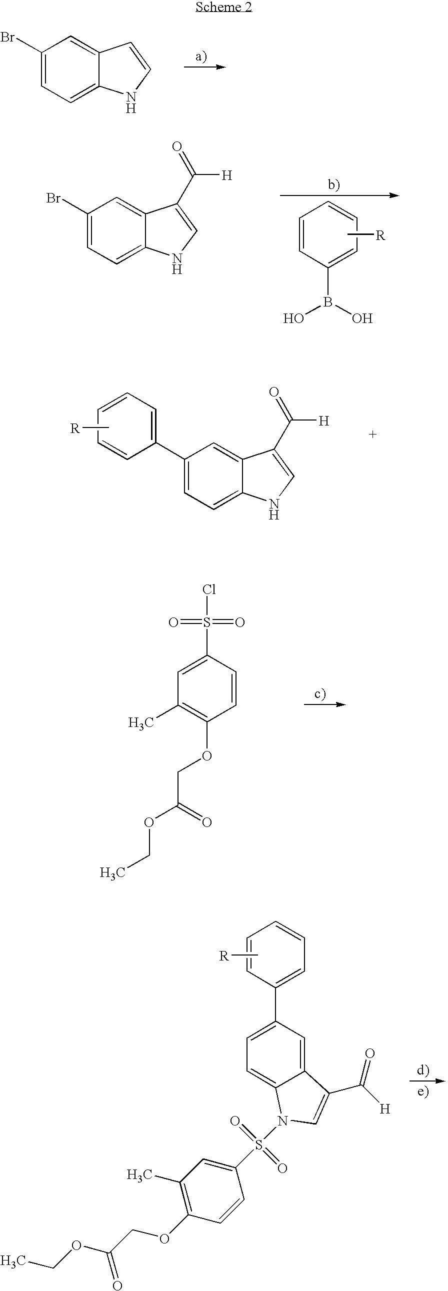

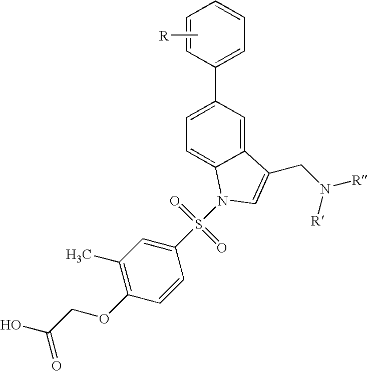

a) SnCl₄, Cl₂CHOCH₃; b) Pd(PPh₃)₂Cl₂, DMF, aq. Na₂CO₃, 100° C. 15 H; c) K₂CO₃, 2-butanone, reflux; d) R′R″NH, sodium triacetoxyborohydride, CH₂Cl₂, 40° C., 2 h; e) aq. NaOH, THF, 1 h, RT.

The inventive compounds exhibit a surprising and valuable pharmacological spectrum of action and can therefore be used as versatile medicaments. In particular, they are suitable for the treatment of coronary heart disease, for the prophylaxis of myocardial infarction and for the treatment of restenosis after coronary angioplasty or stenting. The inventive compounds are preferably suitable for treating arteriosclerosis and hypercholesterolemia, for increasing pathologically low HDL levels and for lowering elevated triglyceride and LDL levels. In addition, they can be used for treating obesity, diabetes, for treating metabolic syndrome (glucose intolerance, hyperinsulinemia, dyslipidemia and hypertension owing to insulin resistance), hepatic fibrosis and cancer.

The novel active ingredients may be administered alone or, if required, in combination with other active ingredients, preferably from the group of CETP inhibitors, antidiabetics, antioxidants, cytostatics, calcium antagonists, antihypertensives, thyroid hormones and/or thyroid mimetics, inhibitors of HMG-CoA reductase, inhibitors of HMG-CoA reductase expression, squalene synthesis inhibitors, ACAT inhibitors, perfusion promoters, platelet aggregation inhibitors, anticoagulants, angiotensin II receptor antagonists, cholesterol absorption inhibitors, MTP inhibitors, aldolase reductase inhibitors, fibrates, niacin, anoretics, lipase inhibitors and PPAR-α and/or PPAR-γ agonists.

The activity of the inventive compounds can be tested, for example, in vitro by the transactivation assay described in the experimental section.

The activity of the inventive compounds can be tested in vivo, for example, by investigations described in the experimental section.

Useful administration forms for the administration of the inventive compounds are all customary administration forms, i.e. oral, parenteral, inhalative, nasal, sublingual, rectal, external, for example transdermal, or local, for example in the case of implants or stents. In the case of parenteral administration, mention should be made in particular of intravenous, intramuscular or subcutaneous administration, for example as a subcutaneous depot. Preference is given to oral or parenteral administration. Very particular preference is given to oral administration.

The active ingredients may be administered alone or in the form of preparations. Preparations suitable for oral administration include tablets, capsules, pellets, coated tablets, pills, granules, solid and liquid aerosols, syrups, emulsions, suspensions and solutions. In this case, the active ingredient has to be present in such an amount that a therapeutic action is achieved. In general, the active ingredient may be present in a concentration of from 0.1 to 100% by weight, in particular from 0.5 to 90% by weight, preferably from 5 to 80% by weight. In particular, the concentration of the active ingredient should be from 0.5 to 90% by weight, i.e. the active ingredient should be present in amounts which are sufficient to attain the dosage range specified.

For this purpose, the active ingredients may be converted to the customary preparations in a manner known per se. This is effected using inert, nontoxic, pharmaceutically suitable carriers, excipients, solvents, vehicles, emulsifiers and/or dispersants.

Examples of excipients include: water, nontoxic organic solvents, for example paraffins, vegetable oils (e.g. sesame oil), alcohols (e.g. ethanol, glycerol), glycols (e.g. polyethylene glycol), solid carriers such as natural or synthetic ground minerals (e.g. talc or silicates), sugars (e.g. lactose), emusifiers, dispersants (e.g. polyvinylpyrrolidone) and lubricants (e.g. magnesium sulfate).

In the case of oral administration, tablets may of course also comprise additives such as sodium citrate together with additives such as starch, gelatin and the like. Aqueous preparations for oral administration may also be admixed with flavor improvers or dyes.

In the case of oral administration, preference is given to administering dosages of from 0.001 to 5 mg/kg, preferably from 0.005 to 3 mg/kg, of bodyweight per 24 hours.

The working examples which follow illustrate the invention. The invention is not restricted to the examples.

Abbreviations:

| | |
|---|---|
| DMF | N,N-dimethylformamide |
| DMSO | dimethyl sulfoxide |
| ESI | electrospray ionization (in MS) |
| HPLC | high-pressure, high-performance liquid chromatography |
| LC-MS | liquid chromatography-coupled mass spectroscopy |
| MS | mass spectroscopy |
| NMR | nuclear magnetic resonance spectroscopy |
| Ph | phenyl |
| RT | room temperature |
| Rₜ | retention time (in HPLC) |
| THF | tetrahydrofuran |
| aq. | aqueous |

LC-MS Methods:
Method 1:

Instrument: Micromass Quattro LCZ, HP1100; column: Symmetry C18, 50 mm×2.1 mm, 3.5 μm; eluent A: acetonitrile+0.1% formic acid, eluent B: water+0.1% formic acid; gradient: 0.0 min 10% B→4.0 min 90% B→6.0 min 90% B; oven: 40° C.; flow rate: 0.5 ml/min; UV detection: 208-400 nm.

Method 2:

Instrument: Micromass Platform LCZ, HP1100; column: Symmetry C18, 50 mm×2.1 mm, 3.5 μm; eluent A: acetonitrile+0.1% formic acid, eluent B: water+0.1% formic acid; gradient: 0.0 min 10% B→4.0 min 90% B→6.0 min 90% B; oven: 40° C.; flow rate: 0.5 ml/min; UV detection: 208-400 nm.

Method 3:

Instrument: Micromass Platform LCZ, HP1100; column: Symmetry C18, 50 mm×2.1 mm, 3.5 μm; eluent A: acetonitrile+0.1% formic acid, eluent B: water+0.1% formic acid; gradient: 0.0 min 10% A→4.0 min 90% A→6.0 min 90% A; oven: 40° C.; flow rate: 0.5 ml/min; UV detection: 208-400 nm.

Method 4:

Instrument: Micromass Platform LCZ, HP1100; column: Symmetry C18, 50 mm×2.1 mm, 3.5 μm; eluent A: acetonitrile+0.5% formic acid, eluent B: water+0.5% formic acid; gradient: 0.0 min 90% A→4.0 min 10% A→6.0 min 10% A; oven: 50° C. flow rate: 0.5 ml/min; UV detection: 208-400 nm.

Method 5:

Instrument: Micromass ZQ; column: Symmetry C18, 50 mm×2.1 mm, 3.5 μm; eluent A: acetonitrile+0.05% formic acid, eluent B: water+0.05% formic acid; gradient: 0.0 min 90% A→3.5 min 10% A→5.5 min 10% A; oven: 50° C.; flow rate: 0.5 ml/min; UV detection: 210 nm.

Method 6:

Instrument: Micromass ZQ; column: Symmetry C18, 50 mm×2.1 mm, 3.5 μm; eluent A: acetonitrile+0.5% formic acid, eluent B: water+0.5% formic acid; gradient: 0.0 min 95% A→4.5 min 10% A→5.5 min 10% A; oven: 50° C.; flow rate: 1 ml/min; UV detection: 210 nm.

### WORKING EXAMPLES

### Example 1

### [4-({3-Isopropyl-5-[4-(trifluoromethyl)phenyl]-1H-indol-1-yl}sulfonyl)-2-methyl-phenoxy]acetic acid

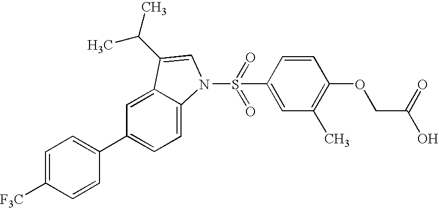 Stage a):

### 1-(4-Bromophenyl)hydrazine

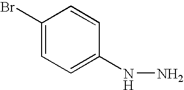

50 g (290.6 mmol) of 4-bromoaniline are heated in 190 ml of concentrated hydrochloric acid to 80° C. for 30 min. After cooling to 5° C., 20 g (290.6 mmol) of sodium nitrite in 95 ml of water are added dropwise over a period of 30 min. After stirring at 5° C. for 30 minutes, the reaction mixture is added dropwise within 45 min to a solution of 384 g (2 mol) of tin chloride in 190 ml of concentrated hydrochloric acid. After a further 45 min at RT, the suspension is made alkaline with 50% sodium hydroxide solution. The precipitate is filtered off and extracted repeatedly with dichloromethane and ethyl acetate. The combined organic phases are dried over magnesium sulfate and concentrated. 37.5 g (68% of theory) of the desired product are obtained.

MS (ESIpos): m/z=186 (M+H)⁺

¹H-NMR (300 MHz, DMSO-d₆): δ=7.01 (d, 2H), 7.26 (d, 2H), 8.18 (s, 2H).

Stage b):

### 5-Bromo-3-isopropyl-1H-indole

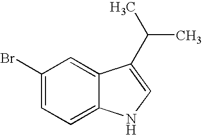

5 g (26.73 mmol) of 1-(4-bromophenyl)hydrazine are suspended in 14 ml of ethanol and admixed with 2.9 g (34.75 mmol) of isovaleraldehyde. After stirring at RT for 30 minutes, the solvent is removed under reduced pressure and the intermediate, without further purification, is fused at 170° C. with 4 g (29.4 mmol) of anhydrous zinc chloride. After 30-45 min, the melt is cooled to RT, taken up in dichloromethane and extracted with dilute hydrochloric acid and water. The organic phase is dried over magnesium sulfate and the solvent is removed under reduced pressure. The crude product is dissolved in ethyl acetate and purified chromatographically on silica gel (eluent: 9:1 cyclohexane/ethyl acetate). 2.7 g (43% of theory) of the desired product are obtained.

LC-MS (method 3): Rₜ=4.9 min.

MS (ESIpos): m/z=238 (M+H)⁺

¹H-NMR (300 MHz, acetone-d₆): δ=1.31 (d, 6H), 3.19 (m, 1H), 7.18 (m, 2H), 7.32 d, (1H), 7.72 (s, 1H).

Stage c):

### Ethyl 2-methylphenoxyacetate

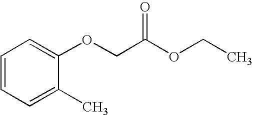

10.81 g (0.10 mol) of 2-methylphenol and 13.82 g (0.10 mol) of potassium carbonate are suspended in 100 ml of N,N-dimethylformamide and stirred at 50° C. for 1 hour. Subsequently, 18.37 g (0.11 mol) of ethyl bromoacetate are added dropwise and the mixture is stirred at 50° C. overnight. After cooling to room temperature, the mixture is concentrated under reduced pressure, taken up with ethyl acetate and washed three times with water. The organic phase is dried over sodium sulfate and freed of solvent under reduced pressure. Distillation of the residue in a Kugelrohr gives 18.5 g (95% of theory) of the desired product.

MS (ESIpos): m/z=194 (M)⁺

¹H-NMR (300 MHz, CDCl₃): δ=1.29 (t, 3H), 2.29 (s, 3H), 4.26 (q, 2H), 4.62 (s, 2H), 6.70 (d, 1H), 6.89 (dt, 1H), 7.22 (t, 1H), 7.25 (d, 1H).

Stage d):

### Ethyl[4-(chlorosulfonyl)-2-methylphenoxy]acetate

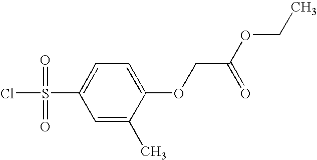

110 g (0.5 mol) of ethyl 2-methylphenoxyacetate are initially charged in 250 ml of chloroform and cooled to 0° C. 330 g (2.8 mol) of chlorosulfonic acid are slowly added dropwise to the solution. After stirring at RT for four hours, the reaction mixture is poured onto ice and extracted three times with dichloromethane. The organic phase is washed twice with water, once with saturated sodium hydrogencarbonate solution and once with saturated sodium chloride solution. After drying over sodium sulfate, the solvent is removed under reduced pressure. 153 g (93% of theory) of the desired product are obtained.

MS (ESIpos): m/z=293 (M+H)⁺

¹H-NMR (300 MHz, CDCl₃): δ=1.31 (t, 3H), 2.36 (s, 3H), 4.28 (q, 2H), 4.75 (s, 2H), 6.81 (m, 2H), 7.85 (m, 2H).

Stage e):

### Ethyl{4-[(5-bromo-3-isopropyl-1H-indol-1-yl)sulfonyl]-2-methylphenoxy}acetate

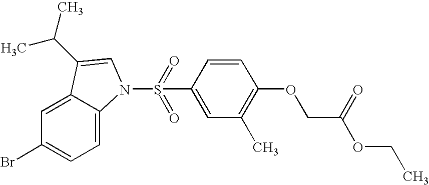

0.10 g (0.42 mmol) of 5-bromo-3-isopropyl-1H-indole are suspended with 0.22 g (0.75 mmol) of ethyl[4-(chlorosulfonyl)-2-methylphenoxy]acetate and 0.17 g (1.26 mmol) of anhydrous potassium carbonate in 5 ml of 2-butanone and heated to reflux for two days. After filtration, the solvent is removed under reduced pressure and the product is purified by means of preparative HPLC (YMC gel ODS-AQ S 5/15 μm; eluent A: water, eluent B: acetonitrile; gradient: 0 min 30% B, 5 min 30% B, 50 min 95% B). 0.14 g (67% of theory) of the desired product is obtained.

LC-MS (method 4): Rₜ=5.59 min.

MS (ESIpos): m/z=494 (M+H)⁺

¹H-NMR (300 MHz, CDCl₃): δ=1.24 (t, 3H), 1.30 (d, 6H), 2.24 (s, 3H), 3.02 (m,

1H), 4.23 (q, 2H), 4.62 (s, 2H), 6.65 (s, 1H), 7.27 (m, 1H), 7.38 (dd, 1H), 7.64 (m, 3H), 7.8 (d, 1H).

Stage f):

[4-({3-Isopropyl-5-[4-(trifluoromethyl)phenyl]-1H-indol-1-yl}sulfonyl)-2-methyl-phenoxy]acetic acid

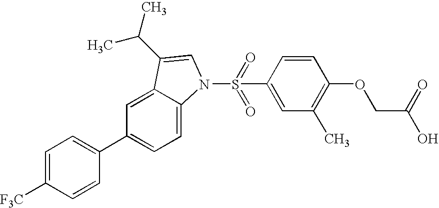

0.09 g (0.18 mmol) of ethyl{4-[(5-bromo-3-isopropyl-1H-indol-1-yl)sulfonyl]-2-methylphenoxy}acetate is dissolved in 6 ml of absolute dimethylformamide and admixed under argon with 6.3 mg (0.009 mmol) of bis(triphenylphosphine)palladium(II) chloride and with 44.9 mg (0.23 mmol) of 4-(trifluoromethyl)phenylboronic acid. After stirring at 70° C. for 30 minutes, 1 ml of 2 M sodium carbonate solution is added. The reaction mixture is heated to 100° C. for 16 h. After cooling to RT, the mixture is filtered through silica gel. The solvent is removed under reduced pressure and the crude product is purified by means of preparative HPLC (YMC gel ODS-AQ S 5/15 μm; eluent A: water, eluent B: acetonitrile; gradient: 0 min 30% B, 5 min 30% B, 50 min 95% B). 60 mg (62% of theory) of the desired product are obtained.

LC-MS (method 4): Rₜ=5.59 min.

MS (ESIpos): m/z=532 (M+H)⁺.

### Example 2

### [4-({3-Acetyl-5-[4-(trifluoromethyl)phenyl]-1H-indol-1-yl}sulfonyl)-2-methyl-phenoxy]acetic acid

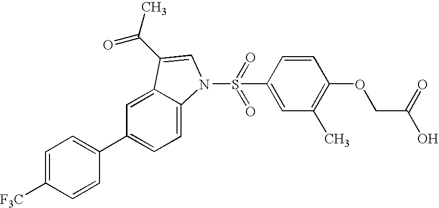 Stage a):

### Ethyl{4-[(3-acetyl-5-bromo-1H-indol-1-yl)sulfonyl]-2-methylphenoxy}acetate

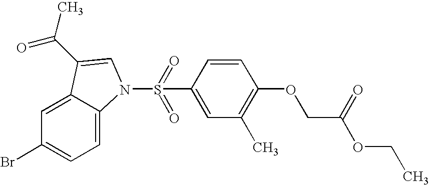

1.2 g (5.04 mmol) of 3-acetyl-5-bromoindole are suspended with 2.6 g (9.07 mmol) of ethyl[4-(chlorosulfonyl)-2-methylphenoxy]acetate and 2.1 g (15.12 mmol) of anhydrous potassium carbonate in 5 ml of 2-butanone and heated under reflux overnight. After filtration, the solvent is removed under reduced pressure and the product is purified by means of preparative HPLC (YMC gel ODS-AQ S 5/15 μm; eluent A: water, eluent B: acetonitrile; gradient: 0 min 30% B, 5 min 30% B, 50 min 95% B). 2.2 g (88% of theory) of the desired product are obtained.

LC-MS (method 5): Rₜ=3.82 min.

MS (ESIpos): m/z=494 (M+H)⁺

¹H-NMR (300 MHz, CDCl₃): δ=1.25 (t, 3H), 2.16 (s, 3H), 2.55 (s, 3H), 4.22 (q, 2H), 4.65 (s, 2H), 6.71 (d, 1H), 7.47 (m, 1H), 7.77 (m, 3H), 8.15 (s, 1H), 8.51 (d, 1H).

Stage b):

### [4-({3-Acetyl-5-[4-(trifluoromethyl)phenyl]-1H-indol-1-yl}sulfonyl)-2-phenoxy]acetic acid

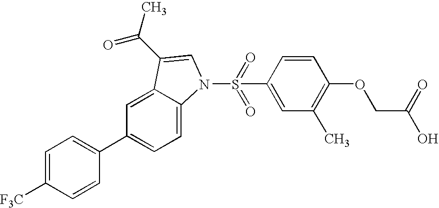

80 mg (0.16 mmol) of ethyl{4-[(3-acetyl-5-bromo-1H-indol-1-yl)sulfonyl]-2-methylphenoxy}acetate are dissolved in 6 ml of absolute dimethylformamide and admixed under argon with 5.6 mg (0.008 mmol) of bis(triphenylphosphine)palladium(II) chloride and with 39.9 mg (0.21 mmol) of 4-(trifluoromethyl)phenylboronic acid. After stirring at 70° C. for 30 min, 1 ml of 2 M sodium carbonate solution is added. The reaction mixture is heated to 100° C. for 16 h. After cooling to RT, the mixture is filtered through silica gel. The solvent is removed under reduced pressure and the crude product is purified by means of preparative HPLC (YMC gel ODS-AQ S 5/15 μm; eluent A: water, eluent B: acetonitrile; gradient: 0 min 30% B, 5 min 30% B, 50 min 95% B). 54 mg (63% of theory) of the desired product are obtained.

LC-MS (method 1): Rₜ=5.3 min.

MS (ESIpos): m/z=532 (M+H)⁺

### Example 3

### [2-Methyl-4-({3-(1,3-thiazol-2-yl)-5-[4-(trifluoromethyl)phenyl]-1H-indol-1-yl}-sulfonyl)phenoxy]acetic acid

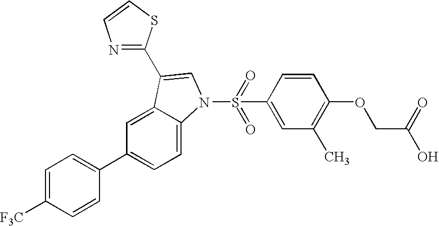 Stage a):

### 5-Bromo-3-(1,3-thiazol-2-yl)-1H-indole

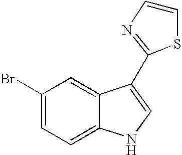

10.2 ml (30.6 mmol) of a 3 M solution of methylmagnesium iodide in diethyl ether are initially charged in 40 ml of absolute toluene and admixed with 5 g (25.5 mmol) of 5-bromoindole in 25 ml of absolute toluene. After stirring at RT for 10 minutes, 2 g (12.7 mmol) of 2-bromothiazole are added dropwise. The reaction mixture is heated to reflux for 6 h, then admixed with water and extracted twice with ethyl acetate. The organic phase is dried over sodium sulfate, filtered and concentrated. The residue is recrystallized from diethyl ether. 1.6 g (22% of theory) of the desired product are obtained.

LC-MS (method 2): Rₜ=4.3 min.

MS (ESIpos): m/z=279 (M+H)⁺

¹H-NMR (300 MHz, DMSO-d₆): δ=7.32 (dd, 1H), 7.46 (d, 1H), 7.56 (d, 1H), 7.83 (d, 1H), 8.15 (s, 1H), 8.40 (d, 1H), 11.9 (s, 1H).

Stage b):

### Ethyl(4-{[5-bromo-3-(1,3-thiazol-2-yl)-1H-indol-1-yl]sulfonyl}-2-methylphenoxy)-acetate

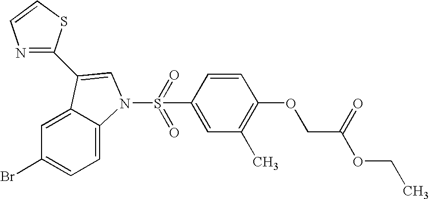

1 g (3.58 mmol) of 5-bromo-3-(1,3-thiazol-2-yl)-1H-indole is suspended with 1.9 g (6.45 mmol) of ethyl[4-(chlorosulfonyl)-2-methylphenoxy]acetate and 1.5 g (10.74 mmol) of anhydrous potassium carbonate in 25 ml of 2-butanone and heated under reflux overnight. After filtration, the solution is admixed with water and extracted with ethyl acetate. After the organic phase has been dried over sodium sulfate, the solvent is removed under reduced pressure and the residue is recrystallized from an ethyl acetate/diethyl ether mixture. 1.3 g (69% of theory) of the desired product are obtained.

LC-MS (method 5): Rₜ=3.76 min.

MS (ESIpos): m/z=535 (M+H)⁺

¹H-NMR (300 MHz, DMSO-d₆): δ=1.14 (t, 3H), 2.19 (s, 3H), 4.11 (q, 2H), 4.93 (s, 2H), 7.03 (d, 1H), 7.61 (dd, 1H), 7.80 (d, 1H), 7.98 (m, 4H), 8.49 (d, 1H), 8.56 (s, 1H).

Stage c):

### [2-Methyl-4-({3-(1,3-thiazol-2-yl)-5-[4-(trifluoromethyl)phenyl]-1H-indol-1-yl}-sulfonyl)phenoxy]acetic acid

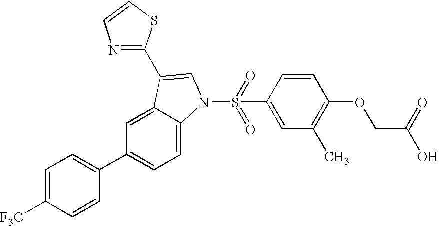

80 mg (0.15 mmol) of ethyl(4-{[5-bromo-3-(1,3-thiazol-2-yl)-1H-indol-1-yl]sulfonyl}-2-methylphenoxy)acetate are dissolved in 6 ml of absolute dimethylformamide and admixed under argon with 5.2 mg (0.007 mmol) of bis(triphenylphosphine)palladium(II) chloride and with 36.8 mg (0.15 mmol) of 4-(trifluoromethyl)phenylboronic acid. After stirring at 70° C. for 30 minutes, 1 ml of 2 M sodium carbonate solution is added. The reaction mixture is heated to 100° C. for 16 h. After cooling to RT, the mixture is filtered through silica gel. The solvent is removed under reduced pressure and the crude product is purified by means of preparative HPLC (YMC gel ODS-AQ S 5/15 μm; eluent A: water, eluent B: acetonitrile; gradient: 0 min 30% B, 5 min 30% B, 50 min 95% B). 71 mg (82% of theory) of the desired product are obtained.

LC-MS (method 2): Rₜ=5.5 min.

MS (ESIpos): m/z=573 (M+H)⁺

¹H-NMR (300 MHz, DMSO-d₆): δ=2.17 (s, 3H), 4.21 (s, 2H), 6.79 (d, 1H), 7.82 (m, 4H), 7.91 (m, 4H), 7.98 (d, 1H), 8.13 (d, 1H), 8.52 (s, 1H), 8.59 (s, 1H).

### Example 4

### [[2-Methyl-4-({3-(4-thiomorpholinylmethyl)-5-[4-(trifluoromethyl)phenyl]-1H-indol-1-yl}sulfonyl)phenoxy]acetic acid

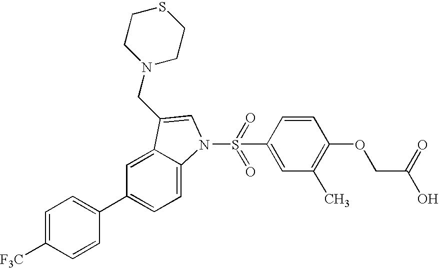 Stage a):

### 5-Bromo-1H-indole-3-carbaldehyde

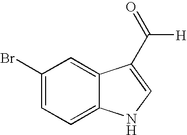

1 g (5 mmol) of 5-bromoindole is initially charged in 3 ml of absolute dichloromethane, blanketed with argon and cooled to −60° C. 2.6 g (10.2 mmol) of tin tetrachloride and 0.7 g (6.1 mmol) of dichloromethyl methyl ether are successively added dropwise. The reaction mixture is warmed to RT, hydrolyzed with 1 N hydrochloric acid and extracted with ethyl acetate. The organic phase is washed repeatedly with water. After the organic phase has been dried over magnesium sulfate, the solvent is removed under reduced pressure and the residue is purified by means of preparative HPLC (YMC gel ODS-AQ S 5/15 μm; eluent A: water, eluent B: acetonitrile; gradient: 0 min 30% B, 5 min 30% B, 50 min 95% B). 0.4 g (32% of theory) of the desired product is obtained.

LC-MS (method 2): Rₜ=4.0 min.

MS (ESIpos): m/z=224 (M+H)⁺

¹H-NMR (300 MHz, acetone-d₆): δ=7.45 (dd, 2H), 8.27 (s, 1H), 8.39 (s, 1H), 10.02 (s, 1H).

Stage b):

### 5-[4-(Trifluoromethyl)phenyl]-1H-indole-3-carbaldehyde

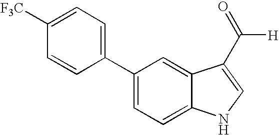

1.4 g (6.3 mmol) of 5-bromo-1H-indole-3-carbaldehyde are dissolved in 60 ml of absolute dimethylformamide and admixed under argon with 0.2 g (0.3 mmol) of bis(triphenylphosphine)palladium(II) chloride and with 1.5 g (8.2 mmol) of 4-(trifluoromethyl)phenylboronic acid. After stirring at 70° C. for 30 minutes, 30 ml of 2 M sodium carbonate solution are added. The reaction mixture is heated to 100° C. for 16 h. After cooling to RT, the mixture is filtered through silica gel. The solvent is removed under reduced pressure and the crude product is purified by means of preparative HPLC (YMC gel ODS-AQ S 5/15 μm; eluent A: water, eluent B: acetonitrile; gradient: 0 min 30% B, 5 min 30% B, 50 min 95% B). 0.37 g (20% of theory) of the desired product is obtained.

LC-MS (method 2): Rₜ=4.7 min.

MS (ESIpos): m/z=290 (M+H)⁺

¹H-NMR (300 MHz, DMSO-d₆): δ=7.64 (s, 2H), 7.82 (d, 2H), 7.91 (d, 2H), 8.36 (s, 1H), 8.43 (s, 1H), 9.99 (s, 1H), 12.25 (s, 1H).

Stage c):

### Ethyl[4-({3-formyl-5-[4-(trifluoromethyl)phenyl]-1H-indol-1-yl}sulfonyl)-2-methyl-phenoxy]acetate

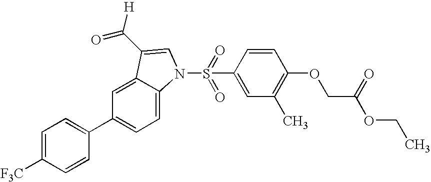

0.35 g (1.3 mmol) of 5-[4-(trifluoromethyl)phenyl]-1H-indole-3-carbaldehyde are suspended with 0.65 g (2.2 mmol) of ethyl[4-(chlorosulfonyl)-2-methylphenoxy]acetate and 0.5 g (3.7 mmol) of anhydrous potassium carbonate in 10 ml of 2-butanone and heated under reflux for 3 h. After filtration, the solvent is removed under reduced pressure and the product is purified by means of preparative HPLC (YMC gel ODS-AQ S 5/15 μm; eluent A: water, eluent B: acetonitrile; gradient: 0 min 30% B, 5 min 30% B, 50 min 95% B). 0.48 g (72% of theory) of the desired product is obtained.

LC-MS (method 2): Rₜ=5.5 min.

MS (ESIpos): m/z=546 (M+H)⁺

¹H-NMR (300 MHz, DMSO-d₆): δ=1.18 (t, 3H), 2.22 (s, 3H), 4.12 (q, 2H), 4.93 (s, 2H), 7.1 (d, 1H), 7.88 (m, 5H), 8.00 (m, 2H), 8.10 (d, 1H), 8.40 (d, 1H), 8.91 (s, 1H), 10.12 (s, 1H).

Stage d):

### Ethyl[2-methyl-4-({3-(4-thiomorpholinylmethyl)-5-[4-(trifluoromethyl)phenyl]-1H-indol-1-yl}sulfonyl)phenoxy]acetate

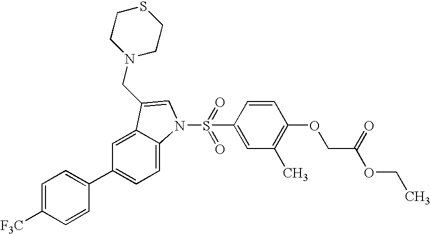

50 mg (0.09 mmol) of ethyl[4-({3-formyl-5-[4-(trifluoromethyl)phenyl]-1H-indol-1-yl}sulfonyl)-2-methylphenoxy]acetate and 9.5 mg (0.09 mmol) of thiomorpholine are initially charged in 3 ml of dichloromethane and admixed with 27.2 mg (0.13 mmol) of sodium triacetoxyborohydride. The reaction mixture is heated to 50° C. overnight. After hydrolysis with sodium hydrogencarbonate solution, the mixture is extracted with dichloromethane. The combined organic phases are washed with saturated sodium chloride solution. After the organic phase has been dried over magnesium sulfate, the solvent is removed under reduced pressure. The product is reacted further without further purification.

LC-MS (method 2): Rₜ=4.2 min.

MS (ESIpos): m/z=633 (M+H)⁺.

Stage e):

### [2-Methyl-4-({3-(4-thiomorpholinylmethyl)-5-[4-(trifluoromethyl)phenyl]-1H-indol-1-yl}sulfonyl)phenoxy]acetic acid

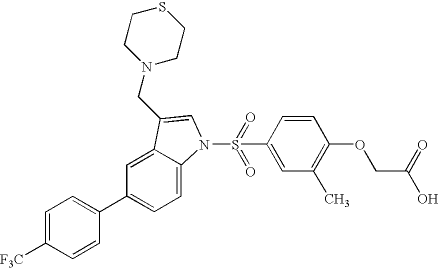

55 mg (0.09 mmol) of ethyl[2-methyl-4-({3-(4-thiomorpholinylmethyl)-5-[4-(trifluoromethyl)phenyl]-1H-indol-1-yl}sulfonyl)phenoxy]acetate are dissolved in 2 ml of tetrahydrofuran and admixed with one drop of 50% sodium hydroxide solution. The reaction mixture is stirred at room temperature for two hours and subsequently hydrolyzed with concentrated hydrochloric acid. After extraction with ethyl acetate, the organic phase is dried over sodium sulfate and the solvent is removed under reduced pressure. The product is purified by means of preparative HPLC (YMC gel ODS-AQ S 5/15 μm; eluent A: water, eluent B: acetonitrile; gradient: 0 min 30% B, 5 min 30% B, 50 min 95% B). 44 mg (83% of theory) of the desired product are obtained.

LC-MS (method 6): Rₜ=2.88 min.

MS (ESIpos): m/z=605 (M+H)⁺

¹H-NMR (300 MHz, DMSO-d₆): δ=2.22 (s, 3H), 2.97 (m, 2H), 3.21 (m, 2H), 3.44 (m, 2H), 3.60 (m, 2H), 4.52 (s, 2H), 4.81 (s, 2H), 7.01 (d, 1H), 7.92 (m, 8H), 8.33 (d, 2H).

The working examples listed in the following table are obtained in an analogous manner:

TABLE 1

| | | Mass | LC- | |
|---|---|---|---|---|
| Ex. | | found | MS Rₜ | LC-MS |
| No. | Structure | [M+H]⁺ | [min.] | method |
| 5 | 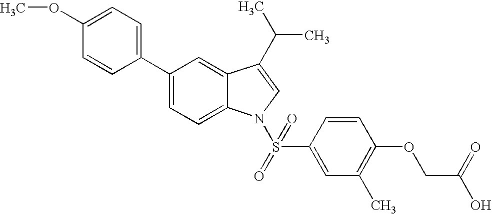 | 494 | 3.4 | 4 |
| 6 | 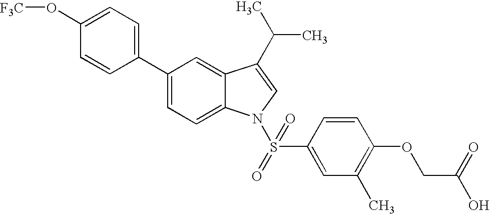 | 548 | 4.71 | 4 |
| 7 | 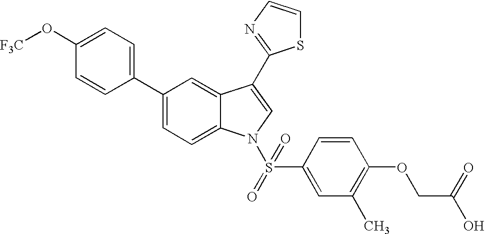 | 589 | 5.6 | 2 |
| 8 | 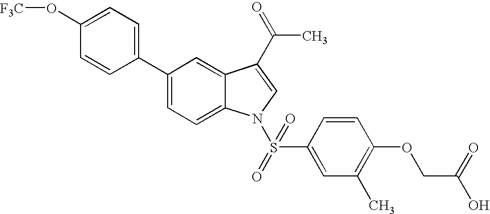 | 548 | 5.3 | 1 |
| 9 | 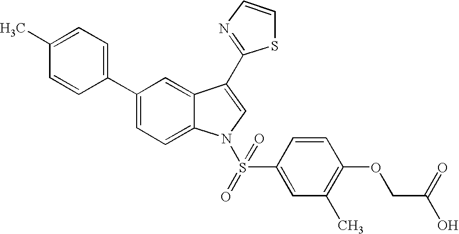 | 519 | 5.4 | 2 |
| 10 | 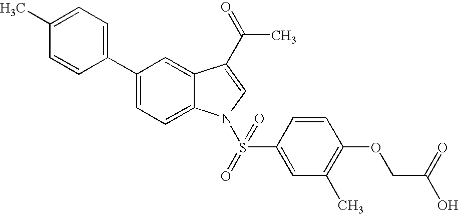 | 478 | 5.2 | 1 |
| 11 | 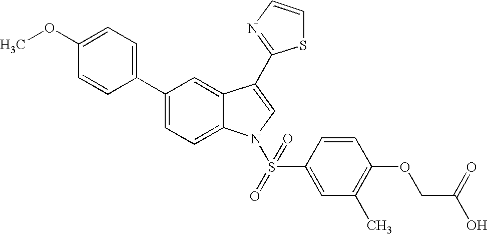 | 535 | 5.1 | 2 |
| 12 | 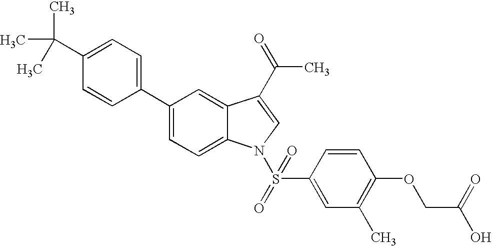 | 520 | 5.6 | 1 |
| 13 | 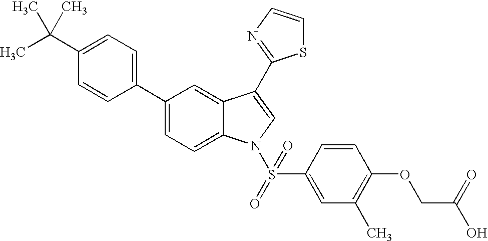 | 561 | 5.9 | 2 |
| 14 | 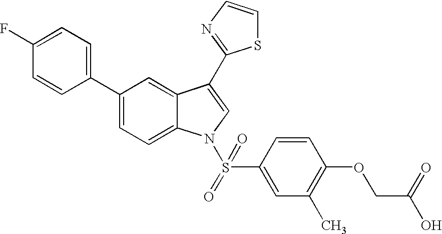 | 523 | 5.2 | 2 |
| 15 | 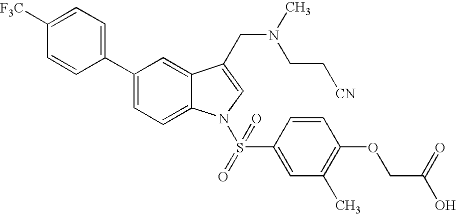 | 586 | 4.2 | 2 |
| 16 | 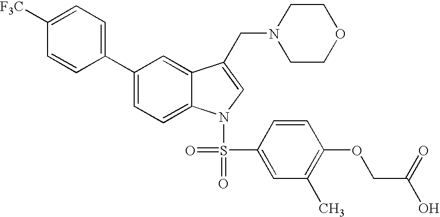 | 589 | 2.71 | 6 |
| 17 | 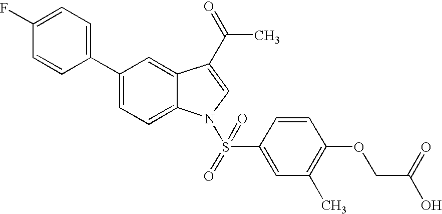 | 482 | 5.0 | 1 |
| 18 | 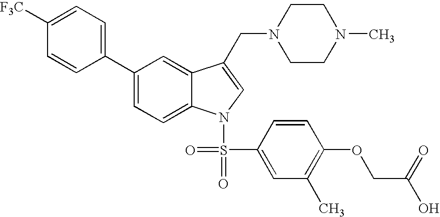 | 602 | 2.84 | 6 |
| 19 | 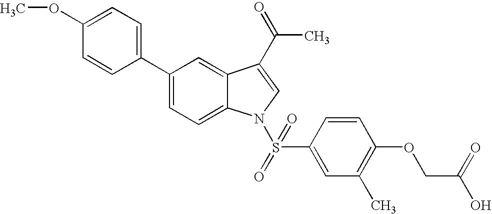 | 494 | 4.91 | 1 |
| 20 | 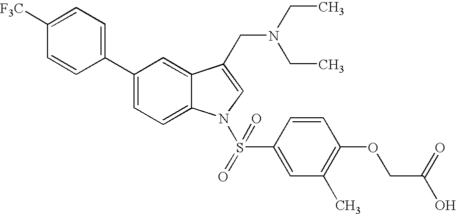 | 575 | 2.7 | 6 |
| 21 | 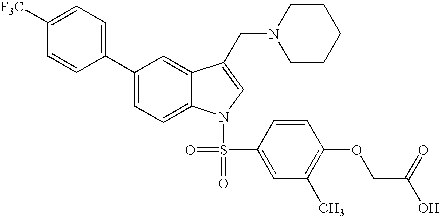 | 586 | 2.73 | 6 |
| 22 | 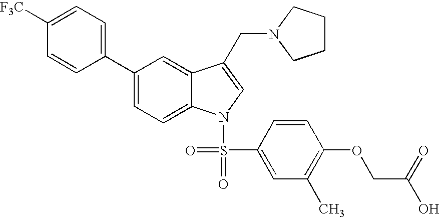 | 573 | 2.73 | 6 |

### Example A

Cellular Transactivation Assay

Test Principle:

A cellular assay is used to identify activators of the peroxisome proliferator-activated receptor delta (PPAR-delta).

Since mammalian cells contain various endogenous nuclear receptors which might complicate an unambiguous interpretation of the results, an established chimera system is used in which the ligand binding domain of the human PPARδ receptor is fused to the DNA binding domain of the yeast transcription factor GAL4. The thus formed GAL4-PPARδ chimera is co-transfected and stably expressed in CHO cells having a reporter construct.

Cloning:

The GAL4-PPARδ expression construct contains the ligand binding domain of PPARδ (amino acids 414-1326), which is PCR-amplified and cloned into the vector pcDNA3.1. This vector already contains the GAL4 DNA binding domain (amino acids 1-147) of the vector pFC2-dbd (Stratagene). The reporter construct, which contains five copies of the GAL4 binding site upstream of a thymidine kinase promoter, expresses firefly luciferase (Photinus pyralis) after activation and binding of GAL4-PPARδ.

Transactivation Assay (Luciferase Reporter):

CHO (chinese hamster ovary) cells are sown in CHO-A-SFM medium (GIBCO), supplemented by 2.5% fetal calf serum and 1% penicillin/streptomycin (G]IBCO), at a cell density of 2×10³ cells per well in a 384-well plate (Greiner). The cells are cultivated at 37° C. for 48 h and then stimulated. To this end, the substances to be tested are taken up in the abovementioned medium and added to the cells. After a stimulation time of 24 hours, the luciferase activity is measured with the aid of a video camera. The relative light units measured give, as a function of the substance concentration, a sigmoidal stimulation curve. The EC₅₀ values are calculated with the aid of the computer program GraphPad PRISM (Version 3.02).

In this test, working examples 1-22 exhibit an EC₅₀ value in a range from 5 nM to 5 μM.

### Example B

Descriptions of the Test for Finding Pharmacologically Active Substances which Increase HDL Cholesterol (HDL-C) Concentrations in the Serum of Transgenic Mice Transfected with the Human ApoA1 Gene (hApoA1) and/or have an Effect on the Metabolic Syndrome of Adipose ob,ob Mice and Lower their Blood Glucose Concentration:

The substances to be examined in vivo for their HDL-C-increasing activity are administered orally to male transgenic hApoA1 mice. One day prior to the start of the experiment, the animals are randomized into groups with the same number of animals, generally n=7-10. Throughout the experiment, the animals have drinking water and feed ad libitum. The substances are administered orally once a day for 7 days. To this end, the test substances are dissolved in a solution of Solutol HS 15+ethanol+saline (0.9%) in a ratio of 1+1+8 or in a solution of Solutol HS 15+saline (0.9%) in a ratio of 2+8. The dissolved substances are administered in a volume of 10 ml/kg of body weight using a stomach tube. Animals which have been treated in exactly the same manner but have only been given the solvent (10 ml/kg of body weight), without test substance, serve as control group.

Prior to the first administration of substance, a blood sample from each of the mice is taken by puncture of the retroorbital venous plexus, to determine ApoA1, serum cholesterol, HDL-C and serum triglycerides (TG) (zero value). Subsequently, using a stomach tube, the test substance is administered for the first time to the animals. 24 hours after the last administration of substance (i.e. on day 8 after the start of the treatment), another blood sample is taken from each animal by puncture of the retroorbital venous plexus, to determine the same parameters. The blood samples are centrifuged and, after the serum has been obtained, cholesterol and TG are determined photometrically using an EPOS Analyzer 5060 (Eppendorf-Gerätebau, Netheler & Hinz GmbH, Hamburg). The determinations is effected using commercial enzyme tests (Boehringer Mannheim, Mannheim).

To determine the HDL-C, the non-HDL-C fraction is precipitated using 20% PEG 8000 in 0.2 M glycine buffer pH 10. From the supernatant, the cholesterol is determined UV-photometrically (BIO-TEK Instruments, USA) in a 96-well plate using a commercial reagent (Ecoline 25, Merck, Darmstadt).

Human mouse-ApoA1 is determined with a Sandwich ELISA method using a polyclonal anti-human-ApoA1 antibody and a monoclonal anti-human-ApoA1 antibody (Biodesign International, USA). Quantification is effected by UV photometry (BIO-TEK Instruments, USA) using peroxidase-coupled anti-mouse-IGG antibodies (KPL, USA) and peroxidase substrate (KPL, USA).

The effect of the test substances on the HDL-C concentration is determined by subtracting the value measured for the 1st blood sample (zero value) from the value measured for the 2nd blood sample (after the treatment). The mean of the differences of all HDL-C values of one group is determined and compared to the mean of the differences of the control group.

Statistical evaluation is carried out using Student's t-test, after the variances have been checked for homogeneity.

Substances which increase the HDL-C of the treated animals in a statistically significant (p<0.05) manner by at least 15%, compared to that of the control group, are considered to be pharmacologically effective.

In order to be able to examine substances for their effect on a metabolic syndrome, animals having an insulin resistance and increased blood glucose levels are used. To this end, C57Bl/6J Lep <ob> mice are treated using the same protocol as for the transgenic ApoA1 mice. The serum lipids are determined as described above. In these animals, serum glucose is additionally determined as a parameter for blood glucose. Serum glucose is determined enzymatically in an EPOS Analyzer 5060 (see above), using commercially available enzyme tests (Boehringer Mannheim).

A blood glucose-lowering effect of the test substances is determined by subtracting the value measured for the 1st blood sample of an animal (zero value) from the value measured for the 2nd blood sample of the same animal (after the treatment). The mean of the differences of all serum glucose values of one group is determined and compared to the mean of the differences of the control group.

Statistical evaluation is carried out using Student's t-test, after the variances have been checked for homogeneity.

Substances which lower the serum glucose concentration of the treated animals in a statistically significant (p<0.05) manner by at least 10%, compared to that of the control group, are considered to be pharmacologically effective.

1. A compound of the general formula (I) 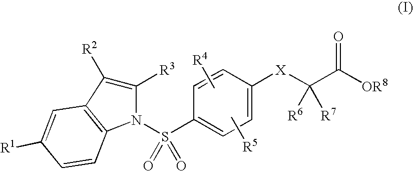
in which
X is O, S or CH₂,
R¹ is (C₆-C₁₀)-aryl or 5- to 10-membered heteroaryl having up to three heteroatoms from the group of N, O and/or S, each of which may be mono- to trisubstituted, identically or differently, by substituents selected from the group of halogen, cyano, nitro, (C₁-C₆)-alkyl which may itself be substituted by hydroxyl or amino, (C₁-C₆)-alkoxy, trifluoromethyl, trifluoromethoxy, (C₂-C₆)-alkenyl, (C₁-C₆)-alkylthio, (C₁-C₆)-alkylsulfonyl, (C₁-C₆)-alkanoyl, (C₁-C₆)-alkoxycarbonyl, hydroxycarbonyl, aminocarbonyl, amino, (C₁-C₆)-acylamino, mono- and di-(C₁-C₆)-alkylamino and 5- to 6-membered heterocyclyl having up to two heteroatoms from the group of N, O and/or S,
R² is phenyl or 5- to 6-membered heteroaryl having up to three heteroatoms from the group of N, O and/or S, each of which may be mono- to trisubstituted, identically or differently, by substituents selected from the group of halogen, cyano, nitro, trifluoromethyl, (C₁-C₄)-alkyl, hydroxyl, trifluoromethoxy and (C₁-C₄)-alkoxy,
or
is (C₁-C₆)-alkyl or (C₁-C₆)-alkanoyl, each of which may be substituted by substituents selected from the group of mono- and di-(C₁-C₆)-alkylamino which may itself be substituted by hydroxyl, amino or cyano, and 5- to 6-membered heterocyclyl which has up to two heteroatoms from the group of N, O and/or S and may itself be substituted by (C₁-C₄)-alkyl,
R³ is hydrogen or (C₁-C₄)-alkyl,
R⁴ is hydrogen or (C₁-C₆)-alkyl,
R⁵ is hydrogen, (C₁-C₆)-alkyl, (C₁-C₆)-alkoxy or halogen,
R⁶ and R⁷ are the same or different and are each independently hydrogen or (C₁-C₄)-alkyl, and
R⁸ is hydrogen or a hydrolyzable group which can be decomposed to the corresponding carboxylic acid,
or a pharmaceutically acceptable salt thereof.

2. A compound of the general formula (I) as claimed in claim 1, in which
X is O or S,
R¹ is phenyl or 5- to 6-membered heteroaryl having up to two heteroatoms from the group of N, O and/or S, each of which may be mono- to disubstituted, identically or differently, by substituents selected from the group of fluorine, chlorine, bromine, cyano, (C₁-C₄)-alkyl, (C₁-C₄)-alkoxy, trifluoromethyl, trifluoromethoxy, methylthio, acetyl, (C₁-C₄)-alkoxycarbonyl, amino, mono- and di-(C₁-C₄)-alkylamino,
R² is phenyl, thiazolyl, oxazolyl, isothiazolyl, isoxazolyl, furyl or thienyl, each of which may be mono- to disubstituted, identically or differently, by substituents selected from the group of fluorine, chlorine, bromine, cyano, nitro, trifluoromethyl, methyl, hydroxyl, methoxy and trifluoromethoxy,
or
is (C₁-C₄)-alkyl or (C₁-C₄)-alkanoyl, each of which may be substituted by substituents selected from the group of di-(C₁-C₄)-alkylamino, pyrrolidino, piperidino, morpholino, thiomorpholino and piperazino, where the heterocycles mentioned may themselves be substituted by (C₁-C₄)-alkyl,
R³ is hydrogen or methyl,
R⁴ is hydrogen or methyl,
R⁵ is hydrogen, (C₁-C₄)-alkyl, (C₁-C₄)-alkoxy, fluorine or chlorine,
R⁶ and R⁷ are the same or different and are each independently hydrogen or methyl,
and
R⁸ is hydrogen.

3. The compound of claim 1, in which
X is O,
R¹ is phenyl which may be mono- to disubstituted, identically or differently, by substituents selected from the group of fluorine, chlorine, methyl, tert-butyl, methoxy, trifluoromethyl, trifluoromethoxy, methylthio and dimethylamino,
R² is thiazolyl, (C₁-C₄)-alkyl, acetyl or a group of the formula —CH₂NR⁹R¹⁰ where
R⁹ and R¹⁰ are the same or different and are each (C₁-C₄)-alkyl, or, together with the nitrogen atom to which they are bonded, form a pyrrolidine, piperidine, morpholine, thiomorpholine, piperazine or N′-methylpiperazine ring,
R³ is hydrogen,
R⁴ is hydrogen or methyl,
R⁵ is methyl,
R⁶ and R⁷ are each hydrogen,
and
R⁸ is hydrogen.

4. A compound of the formula (I-A) 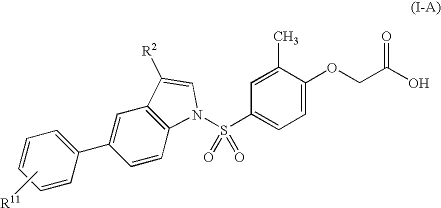
in which
R² is thiazolyi, (C₁-C₄)-alkyl, acetyl or a group of the formula —CH₂NR⁹R¹⁰ where
R⁹ and R¹⁰ are the same or different and are each (C₁-C₄)-alkyl, or, together with the nitrogen atom to which they are bonded, form a pyrrolidine, piperidine, morpholine, thiomorpholine, piperazine or N′-methylpiperazine ring,
and
R¹¹ is fluorine, chlorine, methyl, tert-butyl, trifluoromethyl, methoxy or trifluoromethoxy.

5. A process for preparing a compound of claim 1, characterized in that
compounds of the general formula (II) 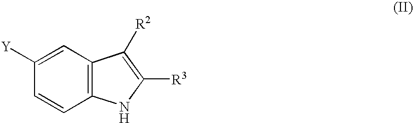
in which R and R are each as defined in claim 1 and
Y is chlorine or bromine,
are converted initially using a compound of the general formula (III) 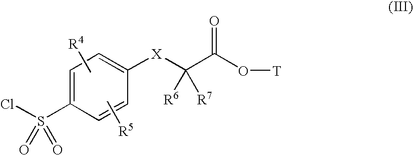
in which X, R⁴, R⁵, R⁶ and R⁷ are each as defined in claim 1 and
T is benzyl or (C-C₆)-alkyl,
in an inert solvent in the presence of a base to compounds of the general formula (IV) 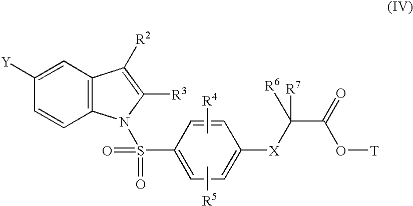
in which T, X, Y, R², R³, R⁴, R⁵, R⁶ and R⁷ are each as defined in claim 1,
then the latter are reacted in a coupling reaction with a compound of the general formula (V) 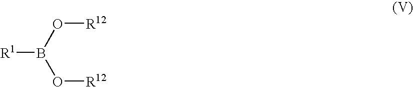
in which R¹ is as defined in claim 1 and
R¹² is hydrogen or methyl, or both radicals together form a —CH₂CH₂— or —C(CH₃)₂—C(CH₃)₂— bridge,
in an inert solvent in the presence of a suitable palladium catalyst and of a base to give compounds of the general formula (I-B) 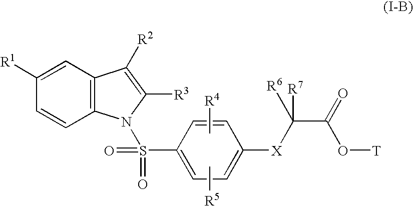
in which T, X, R¹, R², R³, R⁴, R⁵, R⁶ and R⁷ are each as defined in claim 1,
then the compounds (I-B) are reacted with acids or bases or, in the case that T is benzyl, also hydrogenolytically to give the corresponding carboxylic acids of the general formula (I-C) 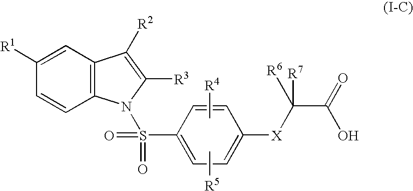
in which X, R¹, R², R³, R⁴, R⁵, R⁶ and R⁷ are each as defined in claim 1,
and the carboxylic acids (I-C) are optionally modified by known methods for esterification further to give compounds of the general formula (I).

6. (canceled)

7. A pharmaceutical composition comprising at least one compound of claim 1 or 4, and inert, nontoxic, pharmaceutically suitable carriers, excipients, solvents, vehicles, emulsifiers or dispersants.

8. (canceled)

9. (canceled)

10. A method of treating or preventing coronary heart diseases and dyslipidemia, preventing myocardial infarction or treating restenosis after coronary angioplasty or stenting, comprising administering to a patient in need thereof a therapeutically effective amount of a compound of claim 1.

11. (canceled)
